(19) United States
(12) Patent Application Publication
(10) Pub. No.: US 2016/0128629 A1
(43) Pub. Date: May 12, 2016

(54) APNEA DETECTION SYSTEM AND METHOD

(71) Applicants: Karen Crow, Santa Fe, NM (US); William Wiecking, Kamuela, HI (US)

(72) Inventors: Karen Crow, Santa Fe, NM (US); William Wiecking, Kamuela, HI (US)

(21) Appl. No.: 14/938,841

(22) Filed: Nov. 11, 2015

Related U.S. Application Data
(60) Provisional application No. 62/078,410, filed on Nov. 11, 2014.

(51) Int. Cl. A61B 5/00; A61B 5/097; A61B 5/0488; A61B 5/08; A61B 5/0476; A61B 5/0496

(52) U.S. Cl. CPC A61B 5/4836; A61B 5/4818; A61B 5/7246; A61B 5/7275; A61B 5/6814; A61B 5/6831; A61B 5/0476; A61B 5/0496; A61B 5/0488; A61B 5/082; A61B 5/0006; A61B 5/0022; A61B 5/097; A61B 2562/06

(57) ABSTRACT

Provided is a method and system to predict a sleep apnea event based upon a sequence of sleep events and mitigate the deleterious effects such an event may have on a person. Some embodiments receive a sequence of sleep events from sensors taking physiological measurements of a person sleeping. These sleep events are compared against grouped sleep apnea events previously gathered from other sleepers with a similar sleep architecture to predict if the person is likely to have a sleep apnea event within an apnea prediction window. If a similar pattern is found, the method sends a sleep apnea warning event responsive to the requested comparison indicating that the person sleeping is likely to experience an imminent sleep apnea event. Various types of sleep entrainment operations may be performed to rouse the person from the sleep to sufficiently to mitigate potential harm from the imminent sleep apnea event.

### CROSS-REFERENCE TO RELATED APPLICATIONS

This application claims priority and is related to U.S. Provisional Application No. 62/078,410, filed Nov. 11, 2014, entitled, “WIRELESS SLEEP APNEA SYSTEM AND METHODS” by William Wiecking and Karen Crow and incorporated by reference herein for all purposes and further related to U.S. Provisional Application No. 62/078,413, filed Nov. 11, 2014, entitled, “SYSTEM AND METHOD FOR GATHERING EXHALED GASES DURING SLEEP” by William Wiecking and Karen Crow and incorporated by reference herein for all purposes.

### TECHNICAL FIELD

The subject matter described herein relates to systems and methods used to monitor and analyze respiration and other bodily functions during sleep. More particularly, the subject matter described herein relates to methods and systems for detecting and then predicting sleep apnea based upon the occurrence of certain events during sleep.

### BACKGROUND

Sleep apnea is a chronic sleep disorder that disrupts regular breathing with either no breathing or in some cases very shallow breathing. Obstructive sleep apnea, the most common type of apnea, occurs when tissue or other structures in the airway, such as tonsils or the tongue, restrict or block air flow to the lungs. As the passageway to the lungs becomes smaller, air passing through the reduced airway can cause the skin tissues to move flutter and generate a snoring sound. Central apnea is another type of apnea caused by a miscommunication between the brain and the muscles used in breathing, and also results in a person shallow breathing or sometimes stopped breathing. Short and long-term effects of sleep apnea may result in high-blood pressure, stroke, heart attacks, heart failure or irregular heart beats or even automobile accidents as drivers suffering from sleep apnea falling asleep at the wheel.

Currently, sufferers from sleep apnea use a Continuous Positive Airway Pressure (CPAP) machine to force air through the nasal cavity and into the lungs in order to keep oxygen in the lungs and prevent the body from suffering from a low or stopped breathing situation. The CPAP device is a relatively noisy device that sits next to or near the individual as they sleep and pumps air with set pressure through a tube and into a mask the individual wears over their face. Depending on the design, and needs of the individual, the mask has straps that go around the back of the head and may cover the nose, the mouth or both the nose and mouth. Some masks are smaller and cover a portion of the face while full-face masks cover the individual's entire face, including their eyes. In some cases, the mask also may include tubes that are placed directly up the nostrils to ensure the nose remains open and oxygen is free to flow through the nasal area.

Because the CPAP device is generally not comfortable and invasive, some individuals may not choose to wear it as frequently and thus defeat its therapeutic effects. It is also possible that the head gear may become dislodged during sleep inadvertently as the individual moves or if the individual subconsciously decides to remove it. It is also possible that wearing the device regularly can further worsen the individuals apnea as the muscles and tissue in the throat lose tone due to the constant pressure being applied by the CPAP device. Accordingly, the individual may become more dependant upon the CPAP device as its continued use further worsens the cause of their sleep apnea in the first instance.

The CPAP device is not only uncomfortable for the individual wearing the device but also may bother others trying to sleep in the same bed or bedroom. In addition to the person snoring while sleeping, the machine may make sounds while pumping air or getting the individual to receive the proper level of oxygen through the mask. Equipment may also take up room in a bedroom and physically interfere with other people sleeping in the same bed.

Sleep apnea remains a disease that affects many people throughout the world but is hard to diagnose accurately and treat. Sleep specialists running sleep labs do not have a standard technique for diagnosing and treating sleep apnea as its occurrence is largely something that occurs in the individual's home. Likewise, the individual does not have a less invasive and more effective method of dealing with sleep apnea other than large, cumbersome and invasive CPAP devices that attempt to continually force air through their nasal cavities and into their lungs.

### SUMMARY

Aspects of the disclosure provide methods, systems, and computer program products for a computer-implemented method of mitigating effects of a sleep apnea event for a person based upon a sequence of sleep events gathered during the person's sleep, comprising receiving a sequence of sleep events from one or more sensor devices taking physiological measurements of a person during sleep, requesting a comparison of the sequence of sleep events from the person against grouped sleep apnea events gathered from other sleepers with a similar sleep architecture to predict if person is likely to have a sleep apnea event within an apnea prediction window, receiving a sleep apnea warning event responsive to the requested comparison indicating that the person associated with the sequence of sleep events is likely to experience an imminent sleep apnea event, performing one or more sleep entrainment operations to rouse the person from the sleep to sufficiently to mitigate potential harm from the imminent sleep apnea event.

Other embodiments include a computer-implemented method of predicting a sleep apnea event for an individual based upon a sequence of sleep events gathered during the person's sleep, comprising receiving a sequence of sleep events from one or more sensor devices gathering physiological measurements on a person during sleep, comparing a pattern from the sequence of sleep events for the person within an apnea prediction window against group sleep apnea events gathered from groups of other sleepers sharing a similar sleep architecture, wherein group sleep apnea events preceded a previously reported apnea event from one or more other people, determining if the pattern from the sequence of sleep events for the person has a similar pattern found in the group sleep apnea events from the one or more other people and transmitting a sleep apnea warning event and associated apnea prediction confidence back to the person through the one or more sensor devices predicting the likelihood of a sleep apnea event occurring in order to mitigate harm to the person from a prospective sleep apnea event

### BRIEF DESCRIPTION OF THE DRAWINGS

FIG. 1 is illustrates a system and method for gathering sleep events and predicting sleep apnea events in accordance with some embodiments;

FIG. 2 illustrates a detailed view of a headband with sensors used to gather sleep event data in accordance with some embodiments;

FIG. 3 is an illustration of a person sleeping with a headband 116 in accordance with some embodiments lying in a comfortable position conducive to sleeping;

FIG. 4 is a flowchart diagram of the operations used to analyze and predict sleep apnea events in accordance with some embodiments;

FIG. 5 is a flowchart diagram illustrating the steps associated with mitigating the effects of sleep apnea event based upon a sequence of sleep events gathered in real-time while a person is sleeping, in accordance with some embodiments; and

FIG. 6 is a block diagram of a computer system used to implement sleep apnea prediction methods and system in accordance with some embodiments.

### DETAILED DESCRIPTION

In the following detailed description, for purposes of explanation, numerous specific details are set forth to provide a thorough understanding of the various embodiments of the disclosure. Those of ordinary skill in the art will realize that these various embodiments are illustrative only and are not intended to be limiting in any way. Other embodiments will readily suggest themselves to such skilled persons having the benefit of this disclosure.

In addition, for clarity purposes, not all of the routine features of the embodiments described herein are shown or described. One of ordinary skill in the art would readily appreciate that in the development of any such actual implementation, numerous implementation-specific decisions may be required to achieve specific design objectives. These design objectives will vary from one implementation to another and from one developer to another. Moreover, it will be appreciated that such a development effort might be complex and time-consuming but would nevertheless be a routine engineering undertaking for those of ordinary skill in the art having the benefit of this disclosure.

A sleep apnea prediction system and method designed in accordance with embodiments offers many advantages and benefits, some of these advantages include one or more of the following.

Various embodiments provide a home based wireless sleep tracking device that synthesizes quality of sleep into a large number of metrics that can be accurately analyzed with reliable results. Cloud-based analysis gathers large amounts of data on the sleep apnea events from many people before making a sleep apnea prediction or diagnosis, and as more people contribute their sleep apnea data over time the results will improve. The sleep apnea prediction is particularly useful in triggering light entrainment or other types of entrainment to rouse the person sleeping and actually avoid the harmful health effects of sleep apnea.

A wireless headband fits like a sweatband and has position sensors to properly fit different individuals and accurately obtain a variety of sensitive sensor data. By designing a comfortable headband, people will likely use the system to gather data nightly thus increasing the data gathered and accuracy of the system in predicting sleep apnea events.

The headband contains dry-sensors and can be positioned by the person sleeping allowing brain activity and other physiological measurements related to sleep to be captured even if user moves around during the night. Because standard wireless communication protocols are implemented, many other sensor devices may be used to provide important sleep event data to the system and method.

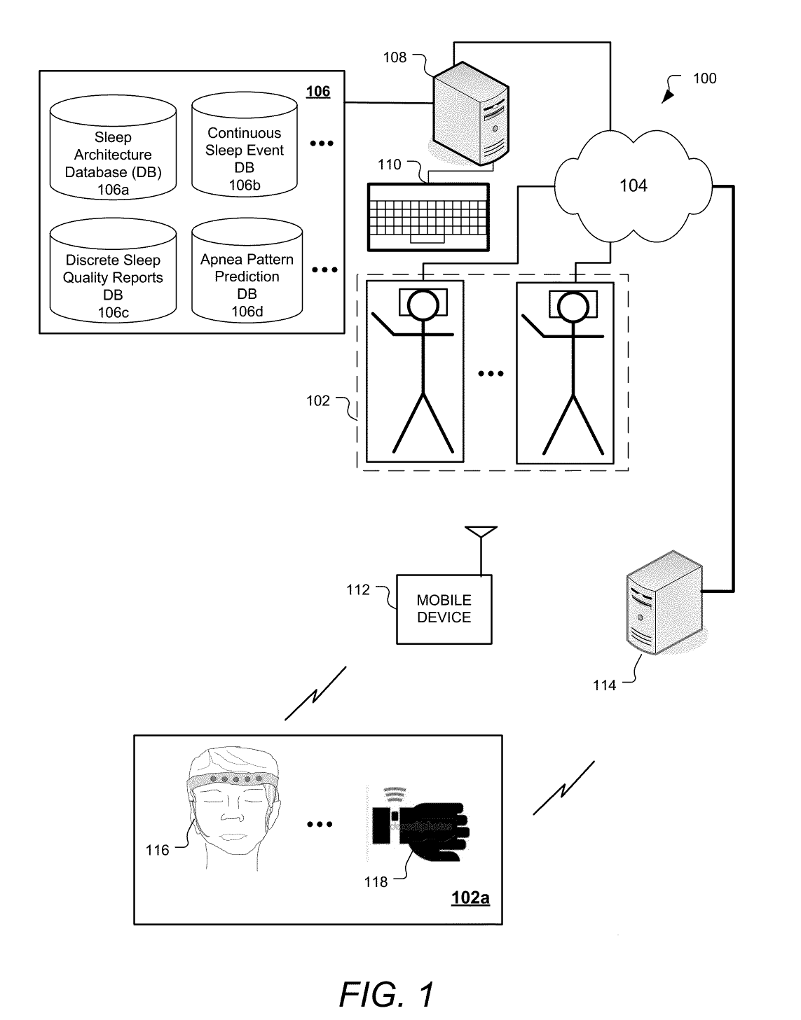

Referring to the figures, FIG. 1 is illustrates a system and method for gathering sleep events and predicting sleep apnea events in accordance with some embodiments. The sleep apnea prediction system 100 in FIG. 1 includes a group of people 102 or sleepers either wearing one or more sensors or having one or more sensors nearby collecting sleep event data as they are sleeping. In some embodiments, people 102 preferably use system 100 on a nightly basis during which their sleep quality may vary from relatively high quality sleep to lower quality sleep interrupted by one or more sleep apnea events. Regardless of the quality of sleep, sensors on or in the vicinity of people 102 gather sleep event data and transmit it for processing over a network 104, such as the Internet, for processing by a server 108 having input devices 110 or cluster of servers running sleep apnea prediction methods in accordance with some embodiments.

All the sleep event data collected over time is stored in Continuous Sleep Event Database (DB) 106b and used for different types of analysis and for reproducing sleep apnea prediction models. When people 102 do actually have a sleep apnea event, the sleep apnea prediction method places a sleep apnea marker into the sequence of sleep events causing the preceding sleep events leading up to the apnea event to be stored into apnea prediction DB 106d for further analysis. Some embodiments will use a variable apnea prediction window to determine how far back in time to store preceding sleep apnea events. This apnea prediction window may start out quite large to support identifying the largest and most complex patterns initially and then later as patterns start to emerge, may reduce the size of the window to a more manageable size and thus decrease the computational time and delay to predict an apnea event. Other embodiments may keep the apnea prediction window as large as possible to give the person sleeping as much time as possible to be experience sufficient entrainment and avoid the sleep apnea event.

To accommodate larger populations of people 102, sleep architecture database 106a may be used to further individualize and make accurate the sleep apnea prediction method and system. Typical sleep architectures progress through a series of four or five types of non-REM and REM cycles (some characterized by rapid eye movement or REM). Each cycle may follow stage-1, stage-2, stage-3, non-REM sleep and then after a period of type in deep stage-3 or slow-wave sleep, return in reverse through stage-3 to stage-2 and then stage-1 type of sleep. Rather than waking, the person may enter a brief period of REM sleep before entering into yet another sleep cycle and this repeats through out the night with the deep stage-3 non-REM sleep decreasing and the REM sleep increasing until waking in the morning.

Since many factors, including sleep apnea itself, can affect the depth and frequency of these cycles, some embodiments will further gather information on people 102 using system 100 to improve prediction accuracy. These sleep architectures may be influenced by people 102 self-reporting information in their profile during registration or sign-up including their age, gender, body mass index (BMI), craniofacial characteristics or other anatomical features determined to affect the architecture. For example, the BMI, being linked to obesity, and certain narrow airways found in some craniofacial structures may both be linked or correlated to increased sleep apnea. Other modifications to sleep architecture may be obtain empirically over time as sleep apnea events from people 102 are gathered and patterns noted. These may include making notations that sleep apnea events are most like to occur during certain REM sleep cycles during the evening or which subsequent REM sleep cycle is likely to be associated with another sleep apnea event. Accordingly, some embodiments may further group apnea predictions from apnea pattern prediction DB 106d by also classifying people 102 and their particular sleep architectures in sleep architecture database 106a.

Sleep apnea prediction system 100 may also include discrete sleep quality reports 106c in another database to hold reports produced manually by sleep specialist reviewing raw sleep event data. These reports are referred to as polysomnography or PSG repots and is a type of sleep study often done in a lab or clinical setting and read by a sleep specialist or doctor. Because the information in the PSG is interpreted by a person, it is inherently subjective and may have varying results and reasoning associated with the analysis. It is nonetheless useful in accordance with some embodiments to use the raw data from these reports in initially training the pattern matching capabilities of the system to recognize sleep apnea events. Later, as more people 102 use the system 100, the necessity of using these discrete sleep quality reports may be less advantageous as the consistent and accurate pattern matching capabilities prove to be more accurate.

Person 102a is using a variety of sensors to gather sleep event including a headband 116 and wrist sensor 118 in accordance with some embodiments. Headband 116 fits over the head much like a sweat band with sensors located near the ears on either side and dry sensors built into the forehead part of the headband for collecting brainwave data. The headband provides flexibility so that the location of the forehead sensors adjusts and sensors are correctly positioned regardless of the size of wearer's head. As a result, the headset can accommodate head sizes from small child to adult. The headset has the ability to integrate other auxiliary sensors, including small CO2 sensor to evaluate respiration rate and exhaled CO2 payload with each breath, motion sensors to detect body motion, jaw clenches, and eye muscle movement and a small infrared sensor to measure pulse and is waterproof to IP66 standards, so can be hand-washed as needed.

Some embodiments may also accept sleep event data from wrist sensor 118 and other consumer health product sensors that attempt to track sleep. Like wrist device 118, consumer health product sensors are typically based on actigraphy or motion detection and sometimes lack accuracy, despite their popularity.

Because embodiments are based upon wireless standards, such as Bluetooth or WiFi, the data from both headband 116 or wrist sensor 118 can be stored locally on a mobile device 112 (i.e., a phone, a tablet or even smart television) as well on a conventional computer 114 for subsequent uploading to servers 108 or other cloud platform associated with sleep apnea detection system 100.

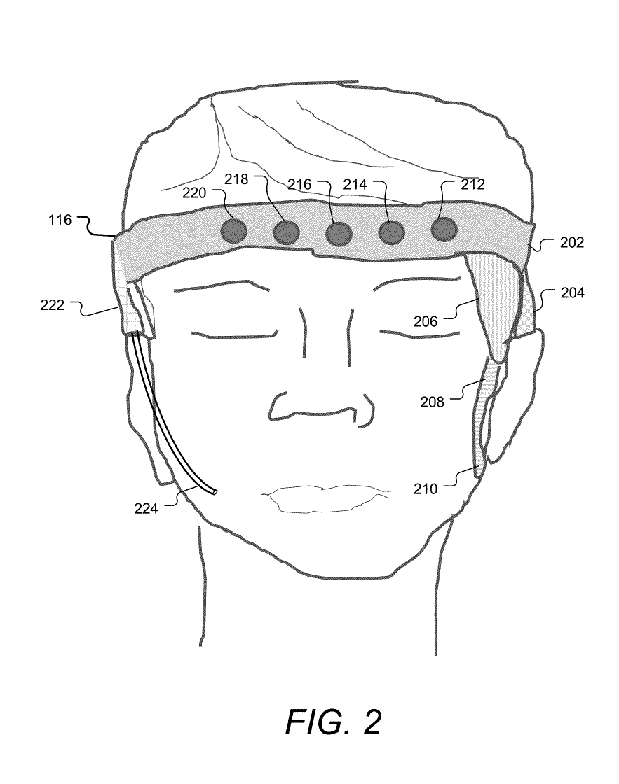

FIG. 2 illustrates a detailed view of a headband 116 with sensors used to gather sleep event data in accordance with some embodiments. In this embodiment, headband 116 includes a non-slip adjustable headband portion 202, a first downward extension 204 with an earpiece having a circuit board, battery, transceiver, electrooculagraphy (EOG) sensor 206, and electromyography (EMG) sensor 210 on EMG extension 208. Some embodiments include electroencephalogram (EEG) 212 and 220 with several position sensors 218, 216 and 214 embedded comfortably in headband portion 202. Second downward extension 222 may also include circuitry, batteries and some sensors as well as a carbon dioxide sensor 224. In practice, the a headband strap portion 220 is configured to be positioned around the forehead region of the person during sleep embedded with position sensors 214, 216, and 218 to indicate positioning of the headband on the forehead. There is also included electroencephalogram (EEG) sensors 212 and 220 to measure brainwaves of the person. On the first downward extension 206 from the headband portion 202, is positioned an electrooculagraphy (EOG) sensor 206 near an eye to detect eye movement and an electromyography (EMG) sensor 210 near muscles of the person's face to detect muscle activity. The second downward extension 222 from the headband portion 202 helps position a carbon dioxide (CO2) sensor near the person's mouth to measure exhaled gasses. In some embodiments, the CO2 sensor incorporates a vacuum assembly having a small volumetric tube that samples the exhaled gases at a rapid frequency and then passes the samples over a high-sensitivity carbon dioxide sensor. And embedded in either the first downward extension 204 and second downward extension 222 is a wireless communications module powered by a portable power supply that transmits the sequence of sleep events wirelessly to a remote computer or mobile device.

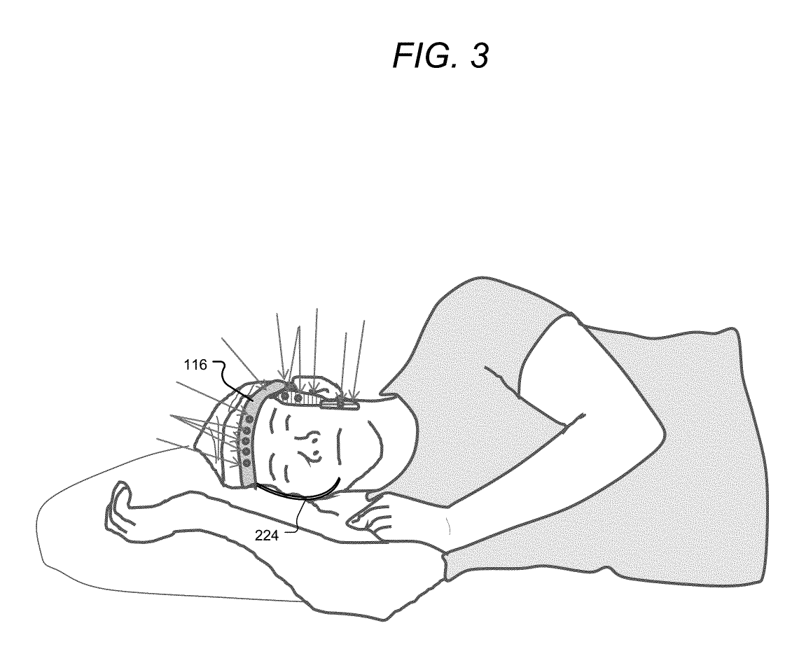

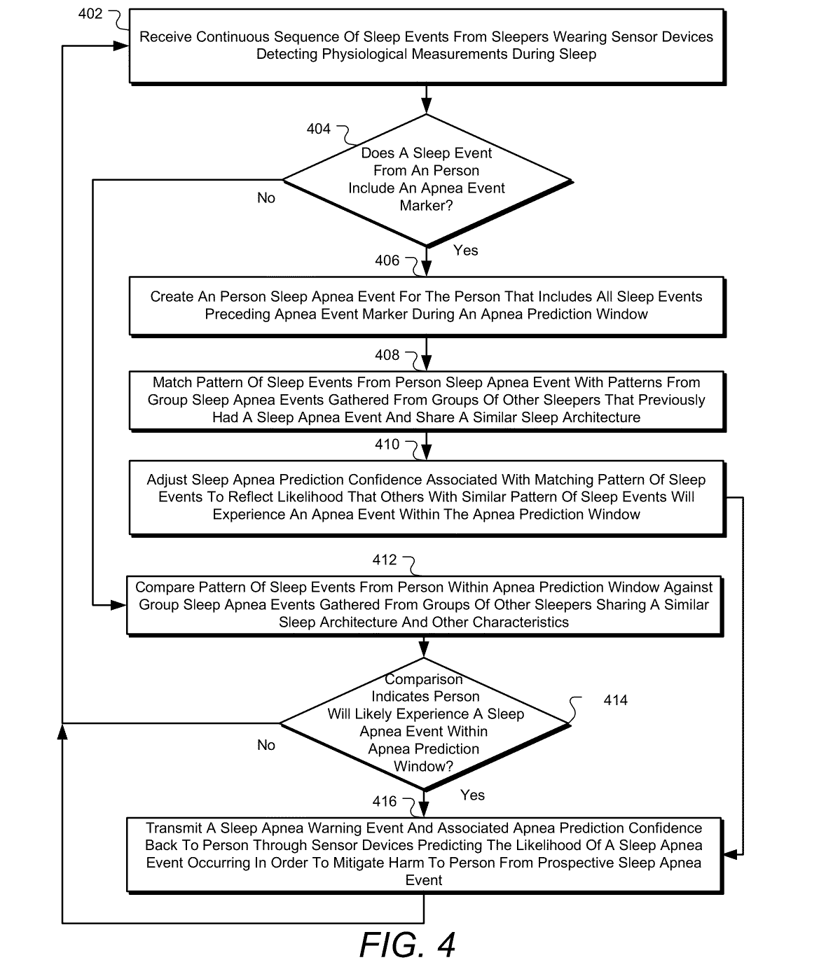

FIG. 3 is an illustration of a person sleeping with a headband 116 in accordance with some embodiments lying in a comfortable position conducive to sleeping. Because the person is unencumbered by wires, with nothing to wake or disturb the person, the person is free to sleep comfortably in any position. FIG. 4 is a flowchart diagram of the operations used to analyze and predict sleep apnea events in accordance with some embodiments. These events are performed on a computer or server device as requested by a person registered with system 100. Initially, the method receives a continuous sequence of sleep events from sleepers wearing sensor devices detecting physiological measurements during sleep (402). Some embodiments, these are sent over a network directly or indirectly, through computers, from one or more sensor devices gathering physiological measurements on a person during sleep.

Next, the method determines if a sleep event from an individual include an apnea event marker (404). The sleep apnea event marker indicates that the person is about to enter into a sleep apnea event and the system should record the preceding sleep events as a record of the sleep apnea event occurring. Accordingly, if the sleep apnea event is present (404—Yes), some embodiments create an individual sleep apnea event for the individual that includes all sleep events preceding apnea event marker during an apnea prediction window (406). This individual sleep apnea event is entered into apnea prediction database db 106d. As previously mentioned, the apnea prediction window determines how far back in time the method should record these events and may vary depending on the type of analysis desired.

The method then matches patterns of sleep events from the individual sleep apnea event with patterns from group sleep apnea events gathered from groups of other sleepers that previously had a sleep apnea event and share similar sleep architecture (408). Accuracy of the apnea prediction method is improved when sleep patterns from individuals having sleep apnea events can be grouped or combined together. This matches the pattern of sleep events from individual sleep apnea event with patterns from group sleep apnea events gathered from groups of other sleepers that previously had a sleep apnea event and share a similar sleep architecture.

Additionally, the method also adjusts a sleep apnea prediction confidence associated with the matching pattern of sleep events to reflect likelihood that others with similar pattern of sleep events will experience an apnea event within the apnea prediction window (410). Matching sleep event patterns that cause sleep apnea acts as a confirmation that the accuracy of the prediction method has improved. Increasing the prediction confidence associated with detected pattern of sleep events reflects the likelihood that others with similar pattern of sleep events will experience an apnea event within the apnea prediction window.

Alternatively, if the person sleeping is not clearly suffering a sleep apnea event, the apnea event market is not part of the sleep events received (404—No). In this case the method attempts to predict a sleep apnea event by comparing a pattern from the sequence of sleep events for the person within an apnea prediction window against group sleep apnea events gathered from groups of other sleepers sharing a similar sleep architecture (412). In some embodiments, this analysis compares the persons sleep events with various group sleep apnea events from a previously reported apnea event from one or more other people.

If there the comparison finds a match (414—Yes), the method indicates the person will likely experience a sleep apnea event within an apnea prediction Window. For example, this apnea prediction window may include a fixed time frame such as one to ten minutes depending on the amount of group sleep apnea events collected from others and the ability to find matching patterns with the person sleeping. When a sleep apnea prediction can be made, the method transmits a sleep apnea warning event and associated apnea prediction confidence back to the person through the one or more sensor devices predicting the likelihood of a sleep apnea event occurring in order to mitigate harm to the person from a prospective sleep apnea event (416). For example, the method may send the sleep apnea warning event to a smartphone or computer located near the person sleeping in order for the computer to kick-off light entrainment or other methods for avoiding the imminent sleep apnea event.

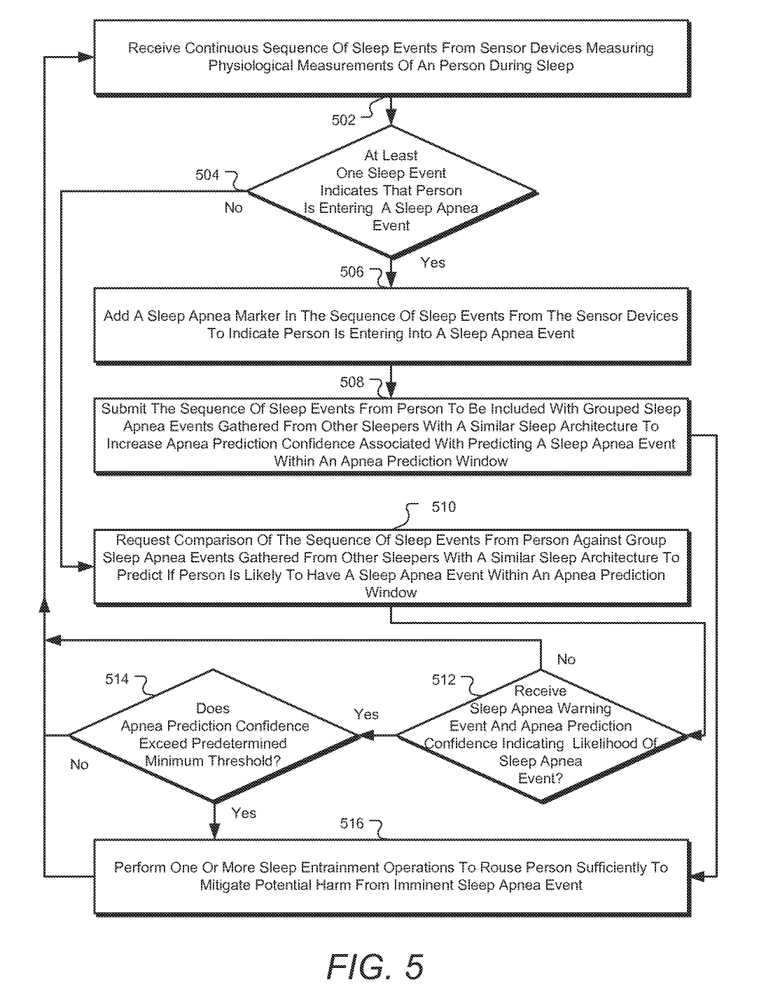

FIG. 5 is a flowchart diagram illustrating the steps associated with mitigating the effects of sleep apnea event based upon a sequence of sleep events gathered in real-time while a person is sleeping, in accordance with some embodiments. In some embodiments, this typically occurs on a remote computer, tablet or device wirelessly connected to the sensors gathering sleep events for the user. Once the person attaches the sensors to their body or in proximity, the method begins receiving a continuous sequence of sleep events from sensor devices measuring physiological measurements of the person (502). Depending on the sleep architecture of the person, the method may also interpret some of the sleep data differently as they enter REM or non-REM sleep.

Next, the method determines if at least one sleep event indicates that person is entering a sleep apnea event. For example, a rapid drop in CO2 gases emitted from the breath of the person sleeping could be a strong indicating that an obstruction has occurred and the person is having difficulty breathing (504—Yes).

Accordingly, the method will add a sleep apnea marker in the sequence of sleep events from the sensor devices to indicate person is entering into a sleep apnea event (506). Next, the method submits the sequence of sleep events from person to be included with grouped sleep apnea events gathered from other sleepers with a similar sleep architecture to increase apnea prediction confidence associated with predicting a sleep apnea event within an apnea prediction window (508). By collecting additional reliable sleep apnea data, the person sleeping may not be able to avoid the sleep apnea event but it contributing to the overall database used for sleep apnea prediction. In some embodiments, the method will still attempt to avoid the apnea event by performing one or more sleep entrainment operations to rouse person sufficiently to mitigate potential harm from imminent sleep apnea event (516).

Alternatively, if no sleep event indicates the person is entering a sleep apnea event (504—No), the method attempts to discover if a sleep apnea event can be predicted. Accordingly, the method then requests comparison of the sequence of sleep events from person against group sleep apnea events gathered from other sleepers with a similar sleep architecture to predict if person is likely to have a sleep apnea event within an apnea prediction window (510). For example, the request may include gathering up a sequence of sleep events and sending over the Internet or other network to be analyzed by a remote server collecting group sleep apnea data.

If the analysis of the sleep event data predicts a sleep apnea event (512—Yes), the method receives a sleep apnea warning event and apnea Prediction confidence indicating likelihood of sleep apnea event. If the apnea prediction confidence exceeds a predetermined threshold (514—Yes), then the method may attempt to avoid the apnea event by performing one or more sleep entrainment operations to rouse person sufficiently to mitigate potential harm from imminent sleep apnea event (516). However, if the apnea prediction confidence is too low to be considered accurate, for example below 10%, then the method may choose to ignore the warning and not engage in a entrainment operation on the person sleeping (514—No)

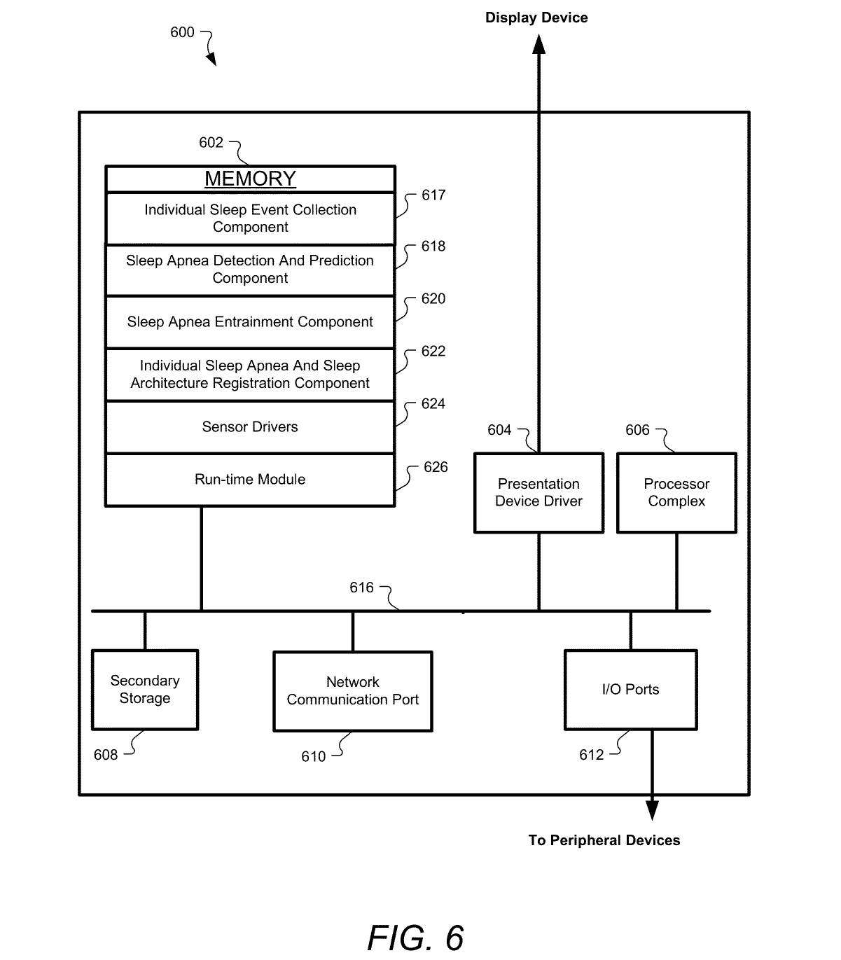

FIG. 6 is a block diagram of a computer system used to implement sleep apnea prediction methods and system in accordance with some embodiments. In this example, the system 600 includes a memory 602 having an individual Sleep event collection component 617, sleep apnea detection and prediction component 618, sleep apnea entrainment component 620, individual sleep apnea and sleep architecture registration component 622, sensor drivers 624 and run-tie module 626. Also included is presentation device driver coupled to a display device, a processor complex 606 for execution of machine instructions, secondary storage 608 for storing data and databases, network communication port 610 to communicate over a network such as the Internet and I/O ports 612 coupled to various other peripheral devices and sensors.

While examples and implementations have been described, they should not serve to limit any aspect of the disclosure. Accordingly, embodiments can be implemented in digital electronic circuitry, or in computer hardware, firmware, software, or in combinations of them. Apparatus of the disclosure can be implemented in a computer program product tangibly embodied in a machine readable storage device for execution by a programmable processor; and method steps of the disclosure can be performed by a programmable processor executing a program of instructions to perform functions by operating on input data and generating output. Embodiments can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high level procedural or object oriented programming language, or in assembly or machine language if desired; and in any case, the language can be a compiled or interpreted language. Suitable processors include, by way of example, both general and special purpose microprocessors. Generally, a processor will receive instructions and data from a read only memory and/or a random access memory. Generally, a computer will include one or more mass storage devices for storing data files; such devices include magnetic disks, such as internal hard disks and removable disks; magneto optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto optical disks; and CD ROM disks. Any of the foregoing can be supplemented by, or incorporated in, ASICs.

While specific embodiments have been described herein for purposes of illustration, various modifications may be made without departing from the spirit and scope of the disclosure. Accordingly, the disclosure is not limited to the above-described implementations, but instead is defined by the appended claims in light of their full scope of equivalents.

1. A computer-implemented method of mitigating effects of a sleep apnea event for a person based upon a sequence of sleep events gathered during the person's sleep, comprising:
receiving a sequence of sleep events from one or more sensor devices taking physiological measurements of a person during sleep;
requesting a comparison of the sequence of sleep events from the person against grouped sleep apnea events gathered from other sleepers with a similar sleep architecture to predict if person is likely to have a sleep apnea event within an apnea prediction window;
receiving a sleep apnea warning event responsive to the requested comparison indicating that the person associated with the sequence of sleep events is likely to experience an imminent sleep apnea event;
performing one or more sleep entrainment operations to rouse the person from the sleep to sufficiently to mitigate potential harm from the imminent sleep apnea event.

2. The computer-implemented method of claim 1 wherein the sequence of sleep events are received continuously at regular time intervals.

3. The computer-implemented method of claim 1 further comprising:
determining if at least one sleep event gathered from the person during sleep indicates that the person is entering a sleep apnea event;
adding a sleep apnea marker into the sequence of sleep events gathered from the person during sleep if it is determined that the person is entering into the sleep apnea event; and
submitting the sequence of sleep events from the person to be included with grouped sleep apnea events gathered from other sleepers with a similar sleep architecture useful in predicting a sleep apnea event.

4. The computer-implemented method of claim 3 wherein the sleep apnea marker identifies the sequence of sleep events as a precursor to a sleep apnea event.

5. The computer-implemented method of claim 3 wherein submitting the sequence of sleep events increases an apnea prediction confidence associated with predicting a sleep apnea event within an apnea prediction window.

6. The computer-implemented method of claim 1 wherein the one or more sensors includes:
a headband strap configured to be positioned around the forehead region of the person during sleep embedded with one or more position sensors to indicate positioning of the headband on the forehead, at least one electroencephalogram (EEG) sensor to measure brainwaves of the person;
a first downward extension from the headband strap that positions an electrooculagraphy (EOG) sensor near an eye to detect eye movement and an electromyography (EMG) sensor near muscles of the person's face to detect muscle activity;
a second downward extension from the headband strap that positions a carbon dioxide (CO2) sensor near the person's mouth to measure exhaled gasses; and
a wireless communications module powered by a portable power supply that transmits the sequence of sleep events wirelessly to a remote computer device.

7. The method of claim 1 wherein requesting the comparison further comprises:
sending the sequence of sleep events over a network to a server that performs an analysis and predicts if the person is likely to have a sleep apnea event.

8. The computer-implemented method of claim 6 wherein the CO2 sensor includes:
a vacuum assembly having a small volumetric tube that samples the exhaled gases at a rapid frequency and then passes the samples over a high-sensitivity carbon dioxide sensor.

9. A computer-implemented method of predicting a sleep apnea event for an individual based upon a sequence of sleep events gathered during the person's sleep, comprising:
receiving a sequence of sleep events from one or more sensor devices gathering physiological measurements on a person during sleep;
comparing a pattern from the sequence of sleep events for the person within an apnea prediction window against group sleep apnea events gathered from groups of other sleepers sharing a similar sleep architecture, wherein group sleep apnea events preceded a previously reported apnea event from one or more other people;
determining if the pattern from the sequence of sleep events for the person has a similar pattern found in the group sleep apnea events from the one or snore other people;
transmitting a sleep apnea warning event and associated apnea prediction confidence back to the person through the one or more sensor devices predicting the likelihood of a sleep apnea event occurring in order to mitigate harm to the person from a prospective sleep apnea event.

10. The computer-implemented method of claim 9, further comprising:
determining if at least one sleep event from the individual includes an apnea event marker, wherein the apnea event marker indicates that the person is about to enter into a sleep apnea event; and
creating an individual sleep apnea event for the individual that includes all sleep events preceding apnea event marker during an apnea prediction window.

11. The computer-implemented method of claim 10, further comprising:
matching the pattern of sleep events from individual sleep apnea event with patterns from group sleep apnea events gathered from groups of other sleepers that previously had a sleep apnea event and share a similar sleep architecture;
combining the individual sleep apnea event with the group sleep apnea events that match; and
adjusting a sleep apnea prediction confidence associated with detected pattern of sleep events to reflect likelihood that others with similar pattern of sleep events will experience an apnea event within the apnea prediction window.

12. A system for mitigating effects of a sleep apnea event for a person based upon a sequence of sleep events gathered during the person's sleep, comprising:
a processor for executing instructions;
a memory for holding instructions when executed on the processor that cause the processor to,
receive a sequence of sleep events from one or more sensor devices taking physiological measurements of a person during sleep;
request a comparison of the sequence of sleep events from the person against grouped sleep apnea events gathered from other sleepers with a similar sleep architecture to predict if the person is likely to have a sleep apnea event within an apnea prediction window;
receive a sleep apnea warning event responsive to the requested comparison indicating that the person associated with the sequence of sleep events is likely to experience an imminent sleep apnea event;
perform one or more sleep entrainment operations to rouse the person from the sleep to sufficiently to mitigate potential harm from the imminent sleep apnea event.

13. The system of claim 12 wherein the sequence of sleep events is received continuously at regular time intervals.

14. The system of claim 12 further comprising instructions when executed on the processor,
determine if at least one sleep event gathered from the person during sleep indicates that the person is entering a sleep apnea event;
add a sleep apnea marker into the sequence of sleep events gathered from the person during sleep if it is determined that the person is entering into the sleep apnea event; and
submit the sequence of sleep events from the person to be included with grouped sleep apnea events gathered from other sleepers with a similar sleep architecture useful in predicting a sleep apnea event.

15. The computer-implemented method of claim 3 wherein the sleep apnea marker identifies the sequence of sleep events as a precursor to a sleep apnea event.

16. The computer-implemented method of claim 3 wherein submitting the sequence of sleep events increases an apnea prediction confidence associated with predicting a sleep apnea event within an apnea prediction window.

17. The computer-implemented method of claim 1 wherein the one or more sensors includes:
a headband strap configured to be positioned around the forehead region of the person during sleep embedded with one or more position sensors to indicate positioning of the headband on the forehead, at least one electroencephalogram (EEG) sensor to measure brainwaves of the person;
a first downward extension from the headband strap that positions an electrooculagraphy (EOG) sensor near an eye to detect eye movement and an electromyography (EMG) sensor near muscles of the person's face to detect muscle activity;
a second downward extension from the headband strap that positions a carbon dioxide (CO2) sensor near the person's mouth to measure exhaled gasses; and
a wireless communications module powered by a portable power supply that transmits the sequence of sleep events wirelessly to a remote computer device.

18. The method of claim 1 wherein requesting the comparison further comprises:
sending the sequence of sleep events over a network to a server that performs an analysis and predicts if the person is likely to have a sleep apnea event.

19. The computer-implemented method of claim 6 wherein the CO2 sensor includes:
a vacuum assembly having a small volumetric tube that samples the exhaled gases at a rapid frequency and then passes the samples over a high-sensitivity carbon dioxide sensor.
